(19) United States
(12) Patent Application Publication
(10) Pub. No.: US 2008/0125712 A1
(43) Pub. Date: May 29, 2008

(54) Ophthalmic injection system

(75) Inventors: Bruno Dacquay, Irvine, CA (US); James Foster, Santa Ana, CA (US); Mark Hopkins, Mission Viejo, CA (US); Lamar Kirchhevel, Laguna Niguel, CA (US); Cesario Dos Santos, Aliso Viejo, CA (US); Salomon Valencia, Aliso Viejo, CA (US)

Correspondence Address: ALCON, IP LEGAL, TB4-8, 6201 SOUTH FREEWAY, FORT WORTH, TX, 76134, US

(73) Assignee: Alcon Manufacturing, Ltd.

(21) Appl. No.: 11/527,245

(22) Filed: Sep. 26, 2006

(51) Int. Cl. A61M 5/155

(52) U.S. Cl. 604/113; 604/143

(57) ABSTRACT

An ophthalmic injection system is disclosed. The system has a dispensing member, a plunger, an actuation chamber, a lock mechanism, a lock stop, a port, a source of pressurized gas, and a shaft. The lock mechanism travels in the actuation chamber in an axial direction toward and away from a distal end of the dispensing member. The lock stop is located on the inner surface of the actuation chamber. The lock stop is engageable with the lock mechanism such that when the lock mechanism travels in the axial direction toward the distal end of the dispensing member to a first position located on a distal side of the lock stop, the lock mechanism cannot travel in a reverse direction away from the distal end of the dispensing member to a location on a proximal side of the lock stop.

### FIELD OF THE INVENTION

The present invention relates to a device for injecting a drug into an eye and more particularly to an ophthalmic drug delivery device with a mechanical lock mechanism.

### BACKGROUND OF THE INVENTION

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually made using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to puncture the eye tissue with the needle, hold the syringe steady, and actuate the syringe plunger (with or without the help of a nurse) to inject the fluid into the eye. The volume injected is typically not controlled in an accurate manner because the vernier on the syringe is not precise relative to the small injection volume. Fluid flow rates are uncontrolled. Reading the vernier is also subject to parallax error. Tissue damage may occur due to an “unsteady” injection. In addition, when the needle is removed from the eye, the drug may be drawn out of the wound if the plunger is retracted. Such reflux leads to imprecise dosing.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. With this type of dispenser, the volumes delivered are highly dependent on fluid viscosity, surface tension, and the specific dispensing tip. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. While precise, this dispenser is expensive and requires an electrical signal to be delivered to the dispensing mechanism.

U.S. Pat. No. 6,290,690 discloses a surgical system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

Despite these efforts, a need remains for a system for injecting precise volumes of substances into the eye without reflux.

### SUMMARY OF THE INVENTION

In one embodiment consistent with the principles of the present invention, the present invention is an ophthalmic injection system that has a dispensing member, a plunger, an actuation chamber, a lock mechanism, a lock stop, a port, a source of pressurized gas, and a shaft. The dispensing member has an inner surface and an outer surface. The inner surface defines a cavity for receiving a quantity of a substance. The plunger is engaged with the inner surface of the dispensing member. The plunger is capable of sliding in the cavity of the dispensing member and is fluidly sealed to the inner surface of the dispensing member. The actuation chamber has an inner surface and an outer surface and encloses a separating member. The separating member has a first end fluidly sealed to the inner surface of the actuation chamber. The lock mechanism is connected to the separating member. The lock mechanism travels in the actuation chamber in an axial direction toward and away from a distal end of the dispensing member. The lock stop is located on the inner surface of the actuation chamber. The lock stop is engageable with the lock mechanism such that when the lock mechanism travels in the axial direction toward the distal end of the dispensing member to a first position located on a distal side of the lock stop, the lock mechanism cannot travel in a reverse direction away from the distal end of the dispensing member to a location on a proximal side of the lock stop. The port connects the inner surface of the actuation chamber to the outer surface of the actuation chamber. The port receives a pressurized gas for moving the separating member. The source of pressurized gas is fluidly coupled to the port. The shaft connects the separating member to the plunger.

In another embodiment consistent with the principles of the present invention, the present invention is an ophthalmic injection system that has a dispensing needle, a plunger, an actuation chamber, a lock mechanism, a lock stop, a port, a source of pressurized gas, and a shaft. The dispensing needle has inner and outer surfaces. The inner surface defines a cavity for receiving a quantity of a drug. The plunger is engaged with the inner surface of the dispensing needle and is capable of sliding in the cavity of the dispensing needle. The plunger is fluidly sealed to the inner surface of the dispensing needle. The actuation chamber has an inner surface and an outer surface, and includes a separating member. The separating member has a first end fluidly sealed to the inner surface of the actuation chamber. The lock mechanism is connected to the separating member. The lock mechanism travels in the actuation chamber in an axial direction toward and away from a distal end of the dispensing member. The lock stop is located on the inner surface of the actuation chamber. The lock stop is engageable with the lock mechanism such that when the lock mechanism travels in the axial direction toward the distal end of the dispensing member to a first position located on a distal side of the lock stop, the lock mechanism cannot travel in a reverse direction away from the distal end of the dispensing member to a location on a proximal side of the lock stop. The port connects the inner surface of the actuation chamber to the outer surface of the actuation chamber and receives a pressurized gas for moving the separating member. The source of pressurized gas is fluidly coupled to the port. The shaft connects the separating member to the plunger.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a prior art syringe.

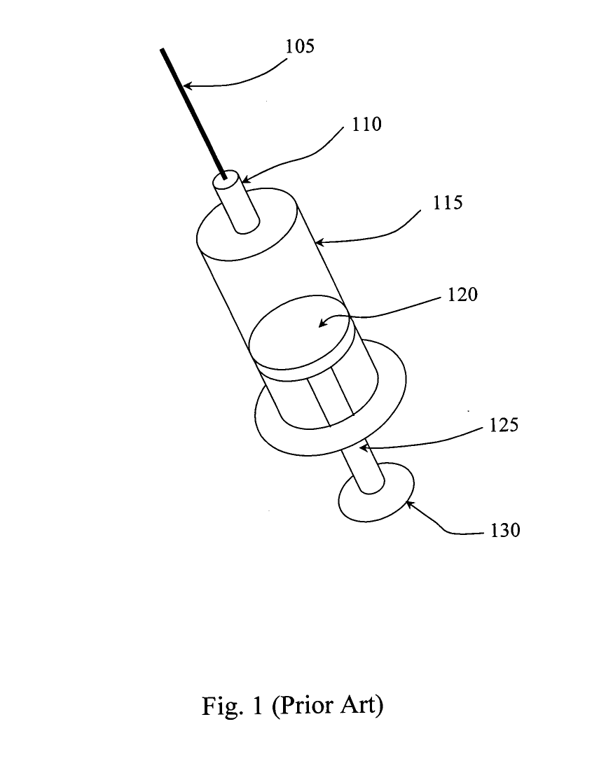

FIG. 2 is a cross section view of an ophthalmic injection system according to an embodiment of the present invention.

FIG. 3 is a cross section view of an ophthalmic injection system according to an embodiment of the present invention.

FIG. 4 is a cross section view of an ophthalmic injection system according to an embodiment of the present invention.

FIG. 5 is a cross section view of an ophthalmic injection system according to an embodiment of the present invention.

FIG. 6 is a cross section view of an ophthalmic injection system according to an embodiment of the present invention.

FIG. 7 is a flow chart of one method of operation according to an embodiment of the present invention.

FIG. 8 is a flow chart of one method of operation according to an embodiment of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

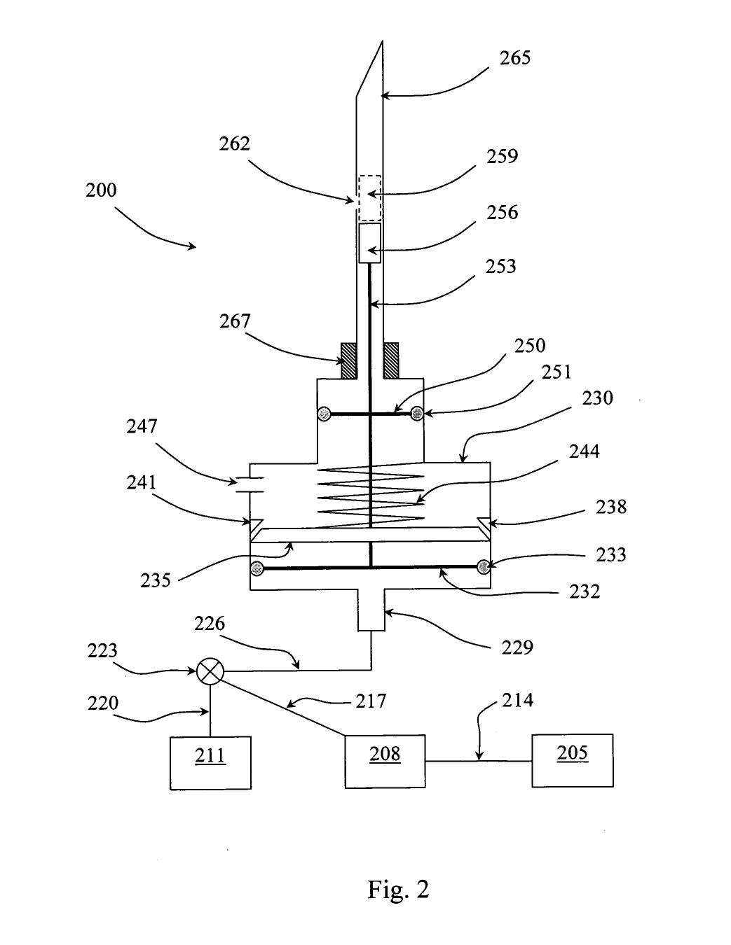

FIG. 2 is a cross section view of an ophthalmic injection system consistent with the principles of the present invention. Ophthalmic injection system 200 generally includes an input control 205, control logic 208, a pressurized gas source 211, a valve 223, an actuation chamber 230, and a dispensing member 265. Actuation chamber 230 has a port 229 and a vent 247, and encloses separating member 232, lock mechanism 235, lock stops 238, 241, spring 244, support 250, and bearing 251. Shaft 253 is located in actuation chamber 230 and in dispensing member 265. Dispensing member has plunger 256, a substance to be injected into an eye 259, and an optional vent 262.

In the embodiment shown in FIG. 2, input control 205 is connected to control logic 208 via interface 214. Control logic 208 is connected to valve 223 via interface 217. Pressurized gas source 211 is fluidly connected to valve 223 via tubing or manifold 220. Valve is fluidly connected to port 229 via tubing or manifold 226.

Shaft 253 connects separating member 232, lock mechanism 235, support 250, and plunger 256. Separating member 232, lock mechanism 235, and support 250 are slidably disposed in actuation chamber 230. Separating member is fluidly sealed to an interior surface of actuation chamber 230. Spring 244 is attached to an interior surface of actuation chamber 230 and to lock mechanism 235.

Dispensing member 265 is connected to actuation chamber 230 via optional luer 267. Plunger 256 is slidably disposed in dispensing member 265 and fluidly sealed to an interior surface of dispensing member 265. The substance to be injected into the eye is located in dispensing member 265 above plunger 256.

Input control 205 is typically a foot switch or other mechanism that can be operated by a medical professional performing the ophthalmic injection. A foot switch is a useful means of actuating the ophthalmic injection system because it leaves the professional's hands free to manipulate the ophthalmic injection system. However, input control 205 may be any type of switch or actuation device to provide an input to the control logic over interface 214.

Control logic 208 is typically one or more integrated circuits that are capable of performing logic functions. Control logic 208 may also be a microprocessor or other similar structure. Control logic 208 receives an input from input control 205 over interface 214 and provides a signal that actuates valve 223 over interface 217. Interfaces 214 and 217 are common electrical or data connectors.

Pressurized gas source 211 provides pressurized gas, such as air or nitrogen, to the system. A manifold or tube 220 allows pressurized gas to travel from pressurized gas source 211 to valve 223. Valve 223 controls the delivery of pressurized gas through tubing or manifold 226 to port 229 and into actuation chamber 230. Valve 223 may be an isolation type valve, proportional type valve, or other type of valve designed to control the flow of pressurized gas from pressurized gas source 211 to port 229.

Actuation chamber 230 is made of a nonpermeable material such as a metal or polymer. Actuation chamber 230 is typically in the shape of a cylinder, though it may be any other shape that has an interior surface. Pressurized gas is introduced into actuation chamber 230 through port 229. Port 229 is located on one end of actuation chamber 230 and is designed to couple with tubing or manifold 226.

Separating member 232 is fluidly sealed to the interior surface of actuation chamber 230 such that pressurized gas entering actuation chamber 230 through port 229 presses against separating member 232 creating a force tending to move separating member 232. If the pressurized gas creates sufficient force against separating member 232, then separating member 232 will slide upward (toward dispensing member 265) in actuation chamber 230.

In FIG. 2, separating member 232 is a piston. In this embodiment, separating member 232 has an end 233 that is fluidly sealed to an interior surface of actuation chamber 230. End 233 may be in the form of an o-ring, washer, or other type of structure that fluidly seals the separating member 232 to an interior surface of actuation chamber 230.

Lock mechanism 235 is rigidly connected to separating member 232 by a shaft 253. Lock mechanism 235 has a beveled cross section as shown. Lock mechanism travels in actuation chamber 230 in a direction along an axis formed by shaft 253. Lock mechanism 235 is generally perpendicular to shaft 253. Lock mechanism 235 is designed to engage and pass over lock stops 238, 241. In this manner, lock mechanism 235 slides in actuation chamber 230 over lock stops 238, 241 to a position on the dispensing member side of the ophthalmic injection system. When in this position, lock mechanism 235 cannot return to its original position on the port side of actuation chamber 230. In other words, lock mechanism 235 slides over lock stops 238, 241 from a position on the port side of actuation chamber 230 to a position on the dispensing member side of actuation chamber 230. When lock mechanism 235 is in this position (on the dispensing member side of lock stops 238, 241), it cannot travel in a reverse direction to a position on the port side of actuation chamber 230 below lock stops 238, 241. In this manner, lock stops 238, 241 prevent lock mechanism 235 from retracting back once it travels past lock stops 238, 241.

Spring 244 provides a biasing or resistive force that pushes against lock mechanism 235. Spring 244 may be replaced with any other means of resistive or biasing force to resist the travel of separating member 232 and lock mechanism 235.

Shaft 253 may be a single integrated part or it may be made up of two or more parts linked together. For example, shaft 253 may be one rigid member. In another embodiment, shaft 253 may be made up of separate smaller rods that are connected together with linkages. In this case, shaft 253 may consist of a first rod connecting separating member 232 to lock mechanism 235, a second rod connecting lock mechanism 235 to support 250, and a third rod connecting support 250 to plunger 256. These three rods may be connected with linkages to form shaft 253.

Support 250 is affixed to shaft 253 or a part thereof. Support 250 has two bearings, such as bearing 251. Support 250 slides inside actuation chamber 230 and provides support for shaft 253. Support 250 may be made of any suitable material.

Dispensing member 265 is typically a needle that consists of a trocar at its terminal end and a cannula for carrying a substance to be injected into an eye. Dispensing member 265 may also be a catheter, lumen or other structure that can facilitate the deposition of a substance into an eye. The terminal or trocar end of dispensing member 265 is open. This allows substance 259 to pass out of the terminal or trocar end of dispensing member 265. Vent 262 in dispensing member 265 is optional. Optional luer 267 connects dispensing member 265 to the remainder of the ophthalmic injection system.

Plunger 256 is located in dispensing member 265. Plunger 256 forms a substantially complete fluid seal with an interior surface of dispensing member 265. In this manner, plunger 256 and dispensing member 265 are very much like a standard syringe.

Substance 259 is typically a drug for treatment of an eye disease or disorder. The drug may be in liquid, partially solid, viscous, or solid form at room temperature.

In operation, a surgeon or medical professional activates input control 205. A signal is sent from input control 205 to control logic 208 via interface 214. Control logic 208 sends a signal to valve 223 via interface 217. This signal causes valve 223 to open allowing pressurized gas from pressurized gas source 211 to pass through tubing or manifold 220, valve 223, tubing or manifold 226, port 229, and into actuation chamber 230. The pressurized gas remains in actuation chamber 230 below separating member 232. As noted, separating member is fluidly sealed to an interior surface of actuation chamber 230 with an o-ring 233 or similar structure. Therefore, pressurized gas builds up in the volume on the port side of separating member 232 bounded by the interior surface of actuation chamber 230 and separating member 232.

When a sufficient force Is applied, separating member 232 is displaced or moved upward and away from port 229. Since separating member 233 is connected to lock mechanism 235 and plunger 256, a displacement of separating member results in a displacement of lock mechanism 235 and plunger 256.

The pressure behind and resulting force necessary to displace separating member 232 is dependent on the resistive or biasing force provided by spring 244. Spring 244 provides a force acting in a direction opposite to the force applied on separating member 232 by the pressurized gas. The force provided by spring 244 is dependent on its spring constant. The spring 244 or other source of resistive or biasing force assists in the control and smooth operation of the ophthalmic injection system. This resistive or biasing force helps to provide a smooth and steady movement of plunger 256 thus allowing a smooth and steady placement of substance 259 into an eye.

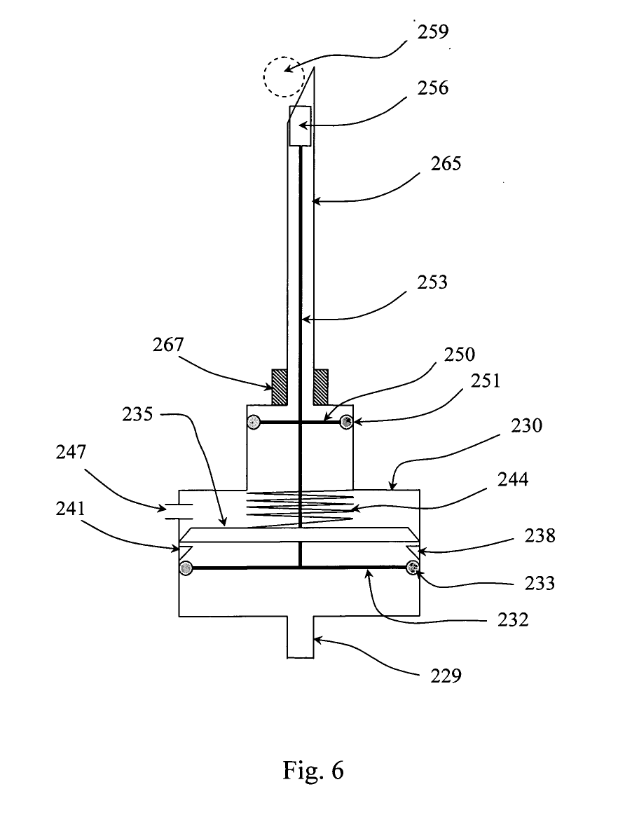

Pressurized gas is applied until the separating member 232, lock mechanism 235, and plunger 256 travel a distance sufficient to deliver substance 259 into an eye. This is more clearly shown in FIG. 6. In FIG. 6, substance 259 has been expelled from dispensing member 265 by plunger 256. In this position, separating member 232 and lock mechanism 235 have traveled upward inside actuation chamber 230. Spring 244 has been compressed. Lock mechanism 235 has traveled past lock stops 238, 241. The beveled edge of lock mechanism 235 contacted the beveled edge of lock stops 238, 241. Sufficient force was applied by the pressurized gas to move lock mechanism 235 beyond lock stops 238, 241. Because of the shape of lock mechanism 235 and lock stops 238, 241, lock mechanism 235 is not able to travel back over lock stops 238, 241. The reverse force provided by spring 244 is not sufficient to cause lock mechanism 235 to travel backwards over lock stops 238, 241. In this manner, lock mechanism can only travel in one direction.

When the source of pressurized gas is no longer applied to port 229, spring 244 provides a force that would tend to move lock mechanism 235 and separating member 232 downward and away from the dispensing member end of ophthalmic injection system 200. As shown in FIG. 6, lock stops 238, 241 prevent lock mechanism 235 from traveling in this reverse direction. In this manner, separating member 232 and plunger 256 remain in position. Plunger 256 does not retract back into dispensing member 265. This prevents reflux of substance 259.

The operation of the ophthalmic injection system allows for precise volumes of a substance to be injected into the eye. It also prevents the substance 259 from being retracted back into the dispensing member 265. The distance that the plunger travels depends on the dosage of the substance 259. The more substance 259 included in dispensing member 265, the further plunger 256 must travel to fully expel the substance. The rate at which the substance 259 is delivered into the eye can be precisely controlled by controlling the pressurized gas and selecting a spring 244 with an appropriate spring constant.

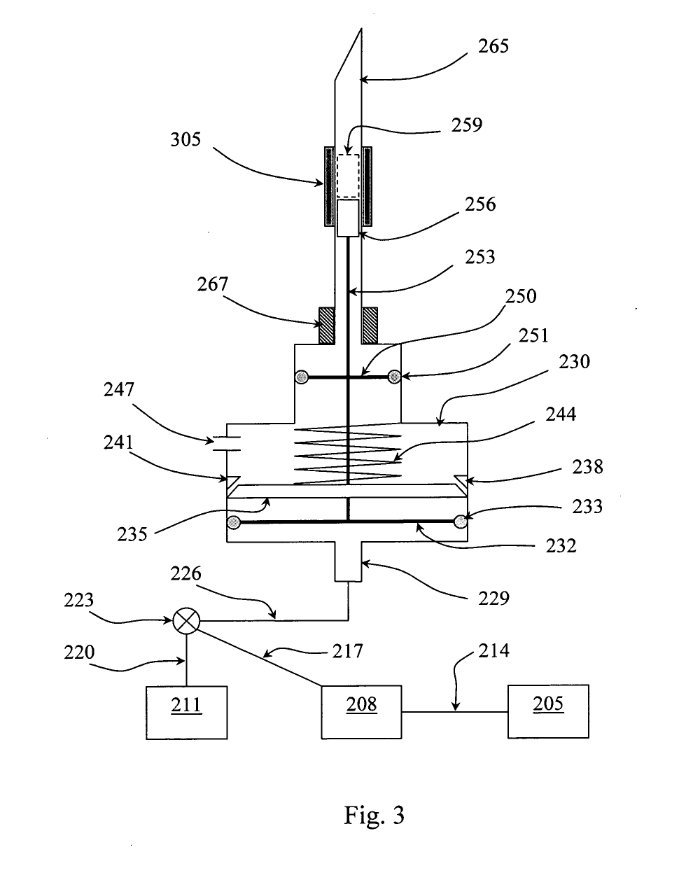

FIG. 3 is a cross section view of an ophthalmic injection system. FIG. 3 has the same structure and operation of FIG. 2 except that FIG. 3 also includes a heater 305. Heater 305 is designed to heat the substance 259 in dispensing member 265. In this embodiment, heater 305 surrounds dispensing member 265. While shown in a location on dispensing member 265, heater 305 may be located on luer 267, a hub to which dispensing needle 265 attaches or any other location such that heater 305 can heat substance 259.

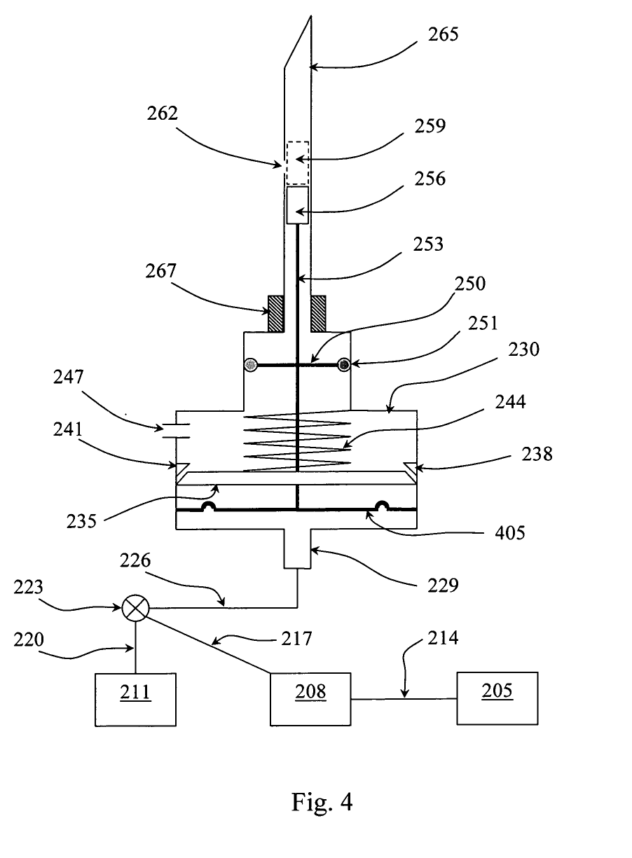

FIG. 4 is a cross section view of an ophthalmic injection system. FIG. 4 has the same structure and operation of FIG. 2, except that in FIG. 4, separating member 232 is depicted as a diaphragm 405. Diaphragm 405 is fluidly sealed to an interior surface of actuation chamber 230. Diaphragm 405 is flexible and can distend to move shaft 256, lock mechanism 235, and plunger 256. When pressurized gas is introduced into actuation chamber 230 through port 229, diaphragm 405 is distended upward in a direction toward dispensing member 265. This movement of diaphragm 405 also moves shaft 253, lock mechanism 235, and plunger 256. The substance 259 is expelled into the eye and the lock mechanism 235 and lock stops 238, 241 operate in the same manner previously described with reference to FIGS. 2 and 6.

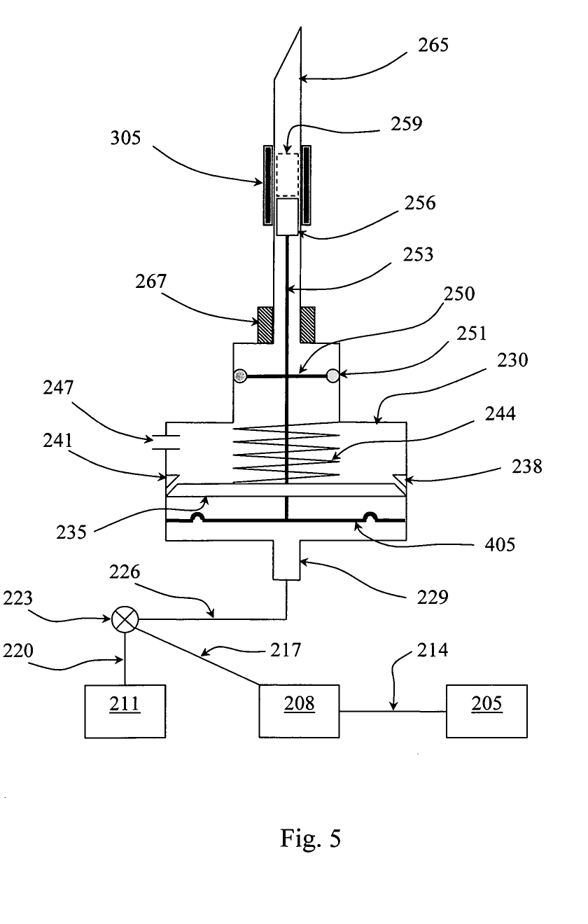

FIG. 5 is a cross section view of an ophthalmic injection system. FIG. 5 has the same structure and operation of FIG. 4 except that FIG. 5 also includes a heater 305. Heater 305 is designed to heat the substance 259 in dispensing member 265. In this embodiment, heater 305 surrounds dispensing member 265. While shown in a location on dispensing member 265, heater 305 may be located on luer 267, a hub to which dispensing needle 265 attaches or any other location such that heater 305 can heat substance 259.

FIG. 6 is a cross section view of an ophthalmic injection system. In FIG. 6, the substance 259 has been expelled into the eye as described above. Lock mechanism 235 is in a locked position in that it cannot travel back over lock stops 238, 241. In this position, the plunger 256 cannot be retracted back into dispensing member 265.

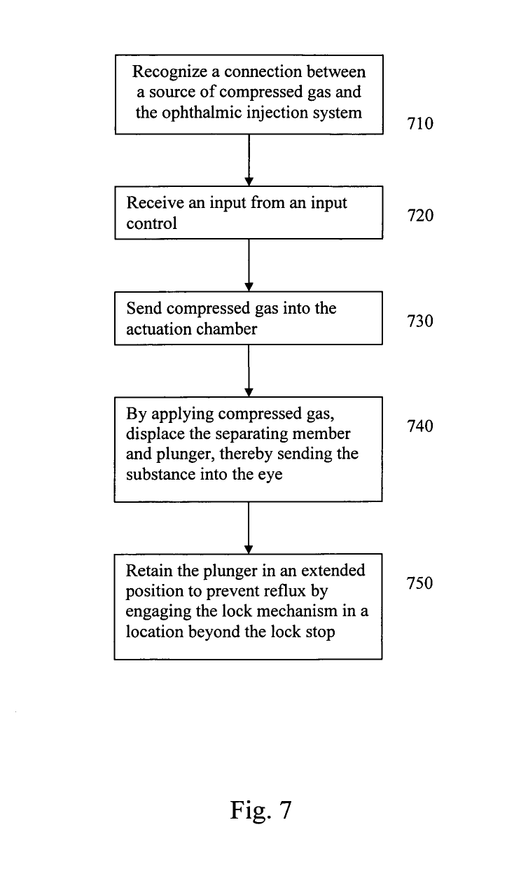

FIG. 7 is a flow chart of one method of operating the ophthalmic injection system consistent with the principles of the present invention. In 710, a connection between a source of compressed gas and the ophthalmic injection system is recognized. In 720, the system receives an input from an input control, such as, for example, a foot switch. In 730, compressed gas is sent into the actuation chamber. In 740, the compressed gas displaces the separating member, thereby moving the plunger and sending the substance into the eye. In 750, the plunger is retained in an extended position to prevent reflux of the substance. The lock mechanism is engaged in a position beyond the lock stops to accomplish this.

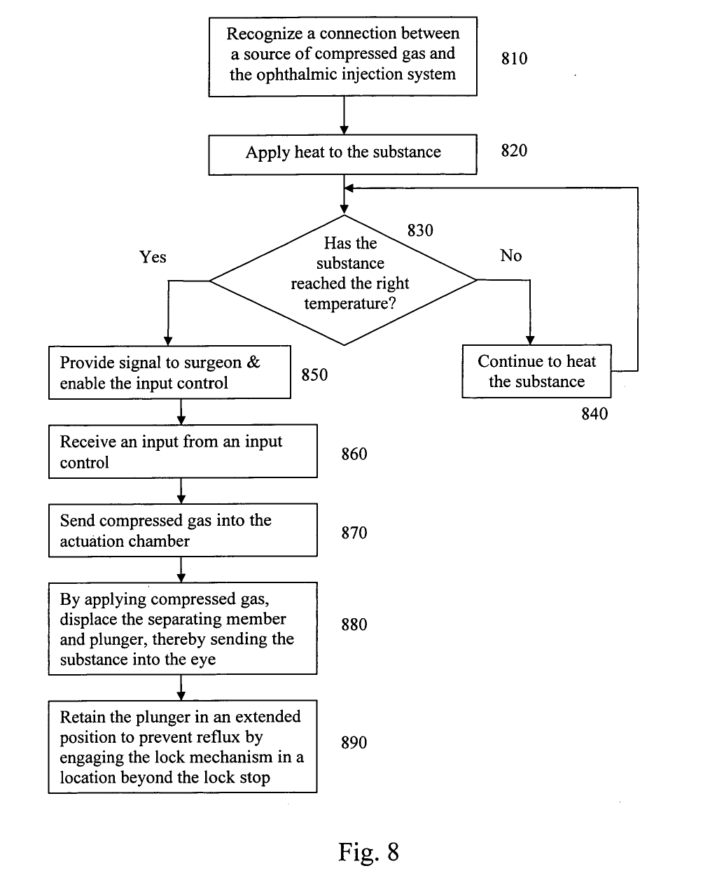

FIG. 8 is a flow chart of one method of operating the ophthalmic injection system consistent with the principles of the present invention. In 810, a connection between a source of compressed gas and the ophthalmic injection system is recognized. In 820, heat is applied to the substance. In 830, if the substance is not heated to the right temperature, then in 840, the substance continues to be heated. If the substance has been heated to the right temperature in 830, then in 850, a signal is provided to the surgeon or medical professional indicating that the substance has been heated. The input control is also enabled. In 860, the system receives an input from the input control, such as, for example, a foot switch. In 870, compressed gas is sent into the actuation chamber. In 880, the compressed gas displaces the separating member, thereby moving the plunger and sending the substance into the eye. In 890, the plunger is retained in an extended position to prevent reflux of the substance. The lock mechanism is engaged in a position beyond the lock stops to accomplish this.

From the above, it may be appreciated that the present invention provides an improved system and methods for delivering precise volumes of a substance into an eye. The present invention prevents reflux when the dispensing member is removed by using a lock mechanism and lock stops. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ophthalmic injection system comprising:
a dispensing member having an inner surface and an outer surface, the inner surface defining a cavity for receiving a quantity of a substance;
a plunger engaged with the inner surface of the dispensing member, the plunger capable of sliding in the cavity of the dispensing member, the plunger fluidly sealed to the inner surface of the dispensing member;
an actuation chamber having an inner surface and an outer surface, the actuation chamber further having a separating member, the separating member having an end fluidly sealed to the inner surface of the actuation chamber;
a lock mechanism connected to the separating member, the lock mechanism traveling in the actuation chamber in an axial direction toward a distal end of the dispensing member,
a lock stop located on the inner surface of the actuation chamber, the lock stop engageable with the lock mechanism such that when the lock mechanism travels in the axial direction toward the distal end of the dispensing member to a first position located on a distal side of the lock stop, the lock mechanism cannot travel in a reverse direction away from the distal end of the dispensing member to a location on a proximal side of the lock stop;
a port connecting the inner surface of the actuation chamber to the outer surface of the actuation chamber, the port for receiving a pressurized gas for moving the separating member;
a source of pressurized gas fluidly coupled to the port; and
a shaft connecting the separating member to the plunger.

2. The system of claim 1 further comprising:
control logic for controlling an amount of the pressurized gas received by the actuation chamber through the port;
whereby the control logic directs the use of the pressurized gas to move the separating member and the plunger to inject the quantity of the substance into an eye.

3. The system of claim 2 wherein the control logic directs the use of the pressurized gas to control a flow rate of the quantity of the substance injected into the eye.

4. The system of claim 2 wherein the substance is a drug for treating a condition of the eye.

5. The system of claim 1 wherein the separating member is a piston.

6. The system of claim 1 wherein the separating member is a diaphragm.

7. The system of claim 1 wherein the dispensing member is a needle.

8. The system of claim 1 wherein the dispensing member is a cannula.

9. The system of claim 1 further comprising a heating mechanism in thermal contact with the dispensing member.

10. The system of claim 1 further comprising a luer engageable with the dispensing member.

11. The system of claim 9 further comprising a heating mechanism in thermal contact with the luer.

12. The system of claim 1 further comprising a resistive element in contact with the lock mechanism.

13. The system of claim 12 wherein the resistive element is a spring.

14. The system of claim 2 further comprising an input control interfaced with the control logic, the input control configured to send a signal to the control logic.

15. The system of claim 14 wherein the input control is a foot switch.

16. An ophthalmic drug delivery system comprising:
a dispensing needle having an inner surface and an outer surface, the inner surface defining a cavity for receiving a quantity of a drug;
a plunger engaged with the inner surface of the dispensing needle, the plunger capable of sliding in the cavity of the dispensing needle, the plunger fluidly sealed to the inner surface of the dispensing needle;
an actuation chamber having an inner surface and an outer surface, the actuation chamber further having a separating member, the separating member having an end fluidly sealed to the inner surface of the actuation chamber;
a lock mechanism connected to the separating member, the lock mechanism traveling in the actuation chamber in an axial direction toward a distal end of the dispensing member,
a lock stop located on the inner surface of the actuation chamber, the lock stop engageable with the lock mechanism such that when the lock mechanism travels in the axial direction toward the distal end of the dispensing member to a first position located on a distal side of the lock stop, the lock mechanism cannot travel in a reverse direction away from the distal end of the dispensing member to a location on a proximal side of the lock stop;
a port connecting the inner surface of the actuation chamber to the outer surface of the actuation chamber, the port for receiving a pressurized gas for moving the separating member;
a source of pressurized gas fluidly coupled to the port; and
a shaft connecting the separating member to the plunger.

17. The system of claim 16 further comprising:
control logic for controlling an amount of the pressurized gas received by the actuation chamber through the port;
whereby the control logic directs the use of the pressurized gas to move the separating member and the plunger to inject the quantity of the drug into an eye.

18. The system of claim 17 wherein the control logic directs the use of the pressurized gas to control a flow rate of the quantity of the drug injected into the eye.

19. The system of claim 16 further comprising a heating mechanism in thermal contact with the dispensing needle.

20. The system of claim 19 wherein the drug has a viscosity at room temperature and the heating mechanism is used to heat the drug to a lower viscosity appropriate for injection into the eye.
